(19) United States
(12) Patent Application Publication
(10) Pub. No.: US 2023/0310341 A1
(43) Pub. Date: Oct. 5, 2023

(54) MINIMIZING SKIN IRRITATION DUE TO SKIN ADHESION PATCHES

(71) Applicant: Medtronic MiniMed, Inc., Northridge, CA (US)

(72) Inventors: Ellis Garai, Woodland Hills, CA (US); Ashwin K. Rao, West Hills, CA (US); Margaret R. Miller, Northridge, CA (US); Sarkis D. Aroyan, Northridge, CA (US)

(73) Assignee: Medtronic MiniMed, Inc., Northridge, CA (US)

(21) Appl. No.: 18/192,479

(22) Filed: Mar. 29, 2023

Related U.S. Application Data
(60) Provisional application No. 63/325,257, filed on Mar. 30, 2022.

(51) Int. Cl. A61K 9/70; A61B 5/145; A61B 5/00

(52) U.S. Cl. CPC A61K 9/7092; A61B 5/14532; A61B 5/6833; A61B 2560/0412

(57) ABSTRACT

The invention provides a skin adhesive patch that inhibits skin irritation by releasing an anti-inflammatory agent such as a corticosteroid topically on the skin surface. Embodiments of the invention can be adapted for use with a wide variety of adhesive patches that are used to couple medical components to the skin of patients such as the devices that diabetic patients use including insulin infusion sets, patch pumps, and all-in-one patch sets.

### CROSS-REFERENCE TO RELATED APPLICATIONS

This application claims the benefit under 35 U.S.C. Section 119(e) of co-pending and U.S. Provisional Patent Application Serial No 63/325,257, filed on Mar. 30, 2022 and entitled “MINIMIZING SKIN IRRITATION DUE TO SKIN ADHESION PATCHES” which application is incorporated by reference herein.

### TECHNICAL FIELD

The invention is directed to adhesive patch systems that are attached to a wearer’s skin.

### BACKGROUND OF THE INVENTION

Adhesive patches are commonly used to attach medical components such as glucose sensors and/or infusion sets to the skin of a user. For example, glucose sensors and/or infusion sets are typically attached to a diabetic wearer’s body to monitor glucose levels and/or deliver insulin from an infusion pump to a subcutaneous site on the wearer’s body. Medical components such as glucose sensors typically comprise a base and a sensor or cannula inserted into an in vivo site on the wearer’s body. In such apparatuses, the base is typically attached to a patch which is attached to the wearer’s body with an adhesive system so that glucose sensors and/or other medical components can be removed and replaced periodically.

Many variations on the adhesive patch are practiced in the art, but in general, the adhesive patch comprises a backing layer, a layer of adhesive adjacent the backing layer which faces the wearer, and a release liner over the adhesive to prevent the adhesive from becoming contaminated before use. Adhesives for this purpose are typically provided commercially already applied to a backing layer, with a release liner covering the adhesive.

As continuous glucose monitoring (CGM) devices move to longer and longer wear durations (e.g., 14-16 days) the probability that a user will experience skin irritation due to the skin adhesive will increase. This skin irritation results in significant patient discomfort and a poor user experience.

In view of the above, new systems and methods designed to minimize skin irritation that occurs due to adhesive patches are desirable.

### SUMMARY OF THE INVENTION

As noted above, as continuous glucose monitoring devices move to longer and longer wear durations the probability that a user will experience skin irritation due to the skin adhesive increases significantly. The invention disclosed herein provides a skin adhesive patch that inhibits skin irritation by releasing an anti-inflammatory agent such as a corticosteroid topically on the skin surface. Embodiments of the invention can be adapted for use with a wide variety of adhesive patches that are used to couple medical components to the skin of patients such as the devices that diabetic patients typically use including glucose sensors, insulin infusion sets, patch pumps, and all-in-one patch (i.e., glucose sensor +insulin pump) sets.

The invention disclosed herein has a number of embodiments. Embodiments of the invention include, for example, an adhesive patch comprising a first flexible layer of material adapted to contact skin, an adhesive composition operatively coupled to the first flexible layer of material, and a therapeutic composition such as a corticosteroid operatively coupled to this first flexible layer of material. In such embodiments, the adhesive composition is adapted to couple the first flexible layer of material to skin; and the therapeutic compound is operatively coupled to the adhesive patch such that when the first flexible layer of material is coupled to skin, the therapeutic compound is exposed to the skin such that inflammation is inhibited at a site on skin where the adhesive patch is adhered. While a wide variety of therapeutic agents can be used in such patches, typically in these embodiments, the therapeutic compound comprises an anti-inflammatory agent such as a corticosteroid.

In typical embodiments of the invention, the adhesive patch is designed to have elements and/or a three-dimensional architecture that facilitates the therapeutic agent diffusing away from the site at which it is disposed and to the skin of a patient (e.g., via conduits or vias that operably connect the therapeutic compound to skin). In some embodiments of the invention, the adhesive composition and/or the therapeutic composition is disposed in the adhesive patch in a selective pattern (e.g, one comprising alternating deposits of adhesive and therapeutic agent). In some embodiments of the invention, the adhesive patch comprises additional elements such as at least one second flexible layer of material (e.g., a second flexible layer also comprising an adhesive composition and which is adapted to adhere the first flexible layer of material to a medical device). Optionally, the therapeutic agent is disposed between the flexible layer of material and the adhesive composition such that the therapeutic agent diffuses through the adhesive composition and to skin; and/or the therapeutic agent is disposed on the adhesive composition such that the therapeutic agent diffuses through the adhesive composition and to skin. In certain embodiments of the invention, the therapeutic compound is disposed on a discrete substrate layer. In typical embodiments of the invention, the adhesive patch is coupled to a medical device such as a glucose sensor or an insulin infusion pump.

Embodiments of the invention include methods of making an adhesive patch as disclosed herein. Typically these methods include operably coupling an adhesive composition to a first flexible layer of material; and also operatively coupling a therapeutic composition to this first flexible layer of material. In such methods, the adhesive composition is adapted to adhere the first flexible layer of material to skin; and the therapeutic compound is operatively coupled to the adhesive patch such that when the first flexible layer of material is adhered to skin, the therapeutic compound is exposed to the skin in a manner such that inflammation is inhibited at a site on skin where the adhesive patch is adhered. In illustrative methods of the invention, the therapeutic compound comprises an anti-inflammatory agent such as a corticosteroid. Certain methods of the invention include the step of coupling the adhesive patch to a medical device.

Embodiments of the invention further include methods for estimating the concentrations of glucose in vivo, the methods comprising disposing an adhesive patch disclosed herein coupled to a glucose sensor on a diabetic patient; and estimating concentrations of glucose using the glucose sensor. Typically in these methods, the adhesive patch is worn by the patient for at least 10 days, at least 15 days, or at least 20 days. Optionally in these methods, the patch is coupled to an insulin infusion set and/or an infusion pump.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 provides schematics showing illustrative embodiments of skin adhesive patches of the invention and systems in which these patches are used. The right panel shows a collapsed/integrated view of a continuous glucose monitoring (CGM) device, and the right panel shows an exploded view of this device illustrating adhesive patch elements. Aspects and embodiments of the invention shown in this figure include a continuous glucose sensor coupled to adhesive patch elements including a double-sided pressure sensitive adhesive and a skin adhesive.

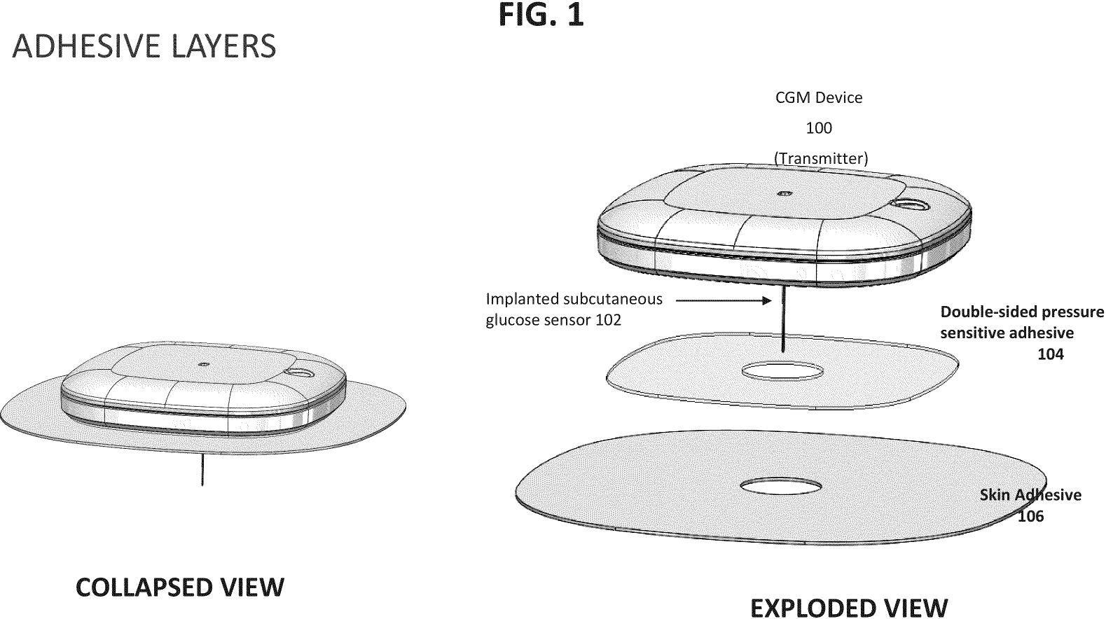

FIG. 2 provides schematics showing illustrative layered embodiments of skin adhesive patches of the invention and systems in which these patches are used. The top panel shows a collapsed/integrated view of a continuous glucose monitoring (CGM) device disposed in vivo, and the bottom panel shows an exploded view of this device illustrating adhesive patch elements. Aspects and embodiments of the invention shown in this figure include a glucose sensor coupled to elements including a backing layer of a skin adhesive (e.g. one formed from nonwoven polyurethane).

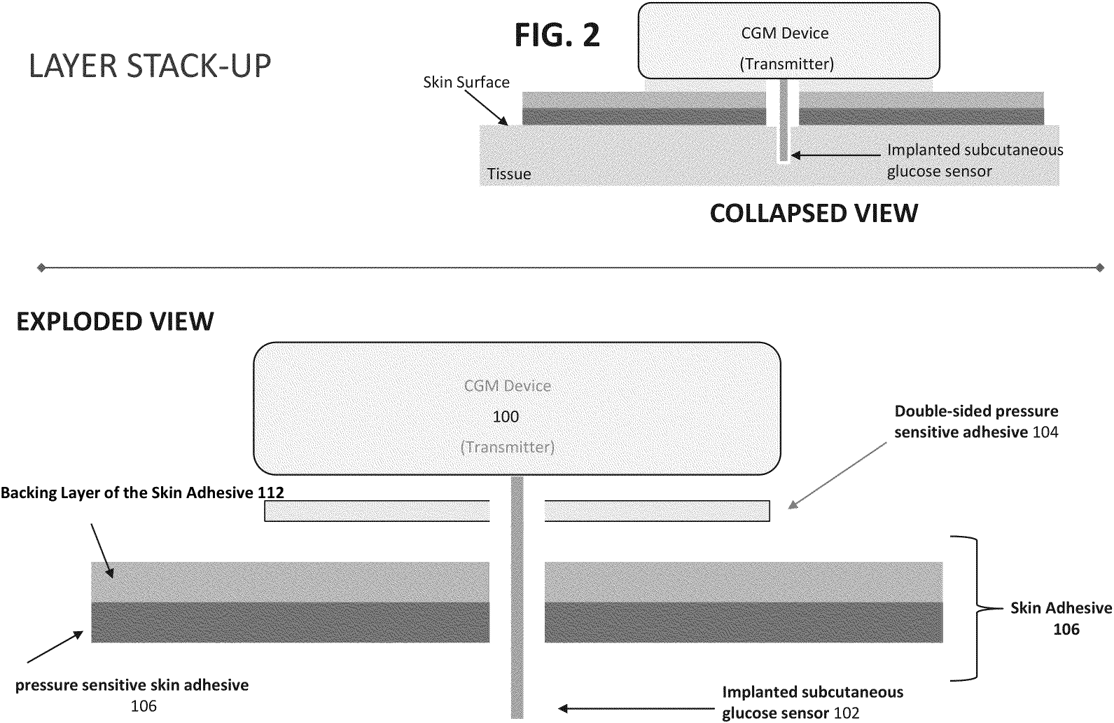

FIG. 3 provides schematics showing illustrative embodiments of skin adhesive patches of the invention and systems in which these patches are used. The top panel shows a collapsed/integrated view of a continuous glucose monitoring (CGM) device disposed in vivo, and the bottom panel shows an exploded view of this device illustrating adhesive patch elements. Aspects and embodiments of the invention shown in this figure include a glucose sensor coupled to elements including various therapeutic agents (e.g., corticosteroids, Zinc Oxide, Neomycin, Clotrimazole, benedryl or the like) as well as patterns of adhesive and therapeutic agent deposition. Topical corticosteroids are frequently used in the treatment of irritant contact dermatitis (ICD). Studies indicate that topical corticosteroids improve healing of ICD. In addition, topical ZnO exhibits strong antioxidant and antibacterial action has been also used in treating atopic dermatitis.

FIG. 4 provides schematics showing illustrative embodiments of skin adhesive patches of the invention and systems in which these patches are used. The top panel shows cross sectional views of an adhesive patch embodiment of the invention, and the bottom panel shows a view of the bottom layer of this skin patch including adhesive and therapeutic (corticosteroid) elements. Aspects and embodiments of the invention shown in this figure include patterns of adhesive and agent deposition with just enough agent/corticosteroid surface area as to not impact adhesive performance. In the bottom panels, a perimeter marking for rigid CGM Device that sits on top of the breathable interface is shown as a dashed line.

FIG. 5 provides schematics showing illustrative embodiments of skin adhesive patches of the invention and systems in which these patches are used. The top panel shows a collapsed/integrated view of a continuous glucose monitoring (CGM) device disposed in vivo, and the bottom panel shows an exploded view of this device illustrating therapeutic agent and adhesive patch elements. Aspects and embodiments of the invention shown in this figure include pressure sensitive skin adhesives and therapeutic agent (corticosteroid) deposition at selected locations in the patch. In this embodiment, therapeutic agent (corticosteroid) diffuses through the adhesive layer.

FIG. 6 provides schematics showing illustrative embodiments of skin adhesive patches of the invention and systems in which these patches are used. The top panel shows a collapsed/integrated view of a continuous glucose monitoring (CGM) device disposed in vivo, and the bottom panel shows an exploded view of this device illustrating therapeutic agent and adhesive patch elements. Aspects and embodiments of the invention shown in this figure include pressure sensitive skin adhesives and therapeutic agent (corticosteroid) deposition at selected locations in the patch. In this embodiment, therapeutic agent (corticosteroid) diffuses through the adhesive layer.

FIG. 7 provides schematics showing illustrative embodiments of skin adhesive patches of the invention and systems in which these patches are used. The top panel shows a collapsed/integrated view of a continuous glucose monitoring (CGM) device disposed in vivo, and the bottom panel shows an exploded view of this device illustrating therapeutic agent and adhesive patch elements. Aspects and embodiments of the invention shown in this figure include layered elements that can be disposed within the patch architecture and patterns of adhesive and agent deposition therein. In this embodiment, therapeutic agent (corticosteroid) diffuses through the adhesive layer.

FIG. 8 provides schematics showing illustrative embodiments of skin adhesive patches of the invention and systems in which these patches are used. The top panel shows a collapsed/integrated view of a continuous glucose monitoring (CGM) device disposed in vivo, and the bottom panel shows an exploded view of this device illustrating therapeutic agent and adhesive patch elements. Aspects and embodiments of the invention shown in this figure include layered elements that can be disposed within the patch architecture, including vias or conduits (microfluidic paths), and patterns of adhesive and agent deposition. In this embodiment, therapeutic agent (corticosteroid) diffuses through the adhesive layer.

FIG. 9 provides schematics showing illustrative embodiments of skin adhesive patches of the invention and systems in which these patches are used. The top panel shows a collapsed/integrated view of a continuous glucose monitoring (CGM) device disposed in vivo, and the bottom panel shows an exploded view of this device illustrating therapeutic agent and adhesive patch elements. Aspects and embodiments of the invention shown in this figure include various layered elements that can be disposed within the patch architecture and patterns of adhesive and agent deposition.

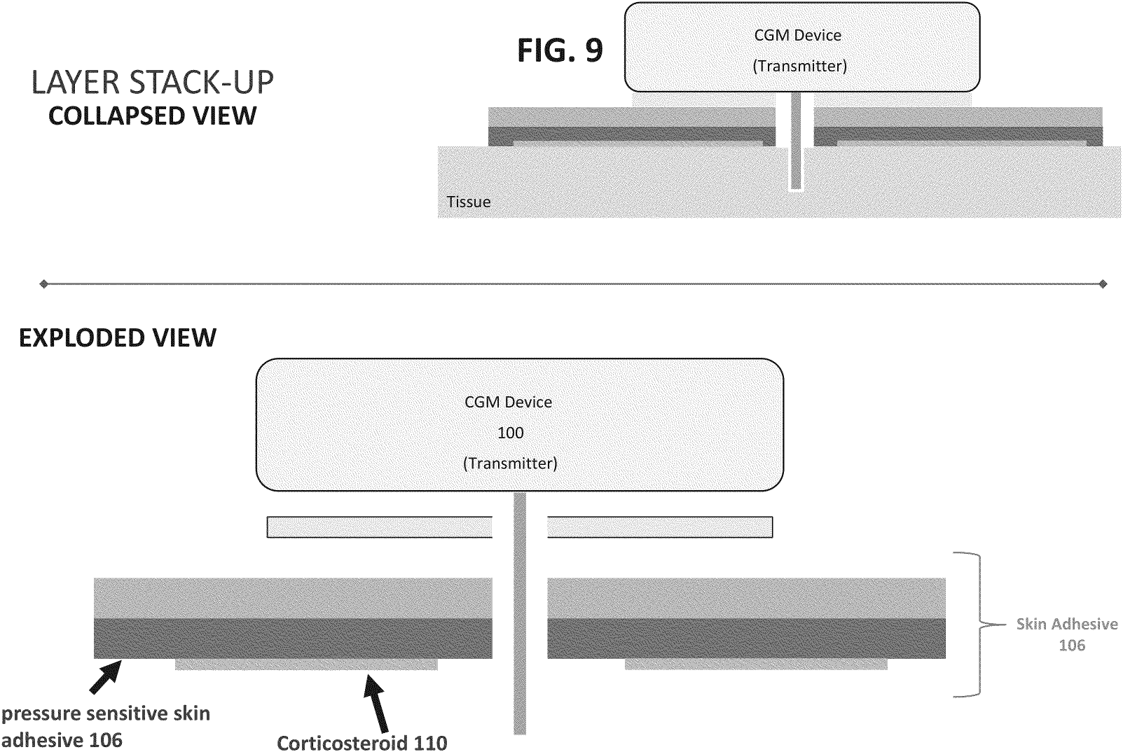

FIG. 10 provides schematics showing illustrative embodiments of skin adhesive patches of the invention and systems in which these patches are used. The top panel shows cross sectional views of an adhesive patch embodiment of the invention, and the bottom panel shows a view of the bottom layers of skin patches including adhesive and therapeutic (corticosteroid) elements disposed in selected patterns. Aspects and embodiments of the invention shown in this figure include illustrative patterns for adhesive and agent deposition with just enough agent/corticosteroid deposited as to not impact adhesive performance. In the bottom panels, a perimeter marking for rigid CGM device that sits on top of the breathable interface is shown as a dashed line.

FIG. 11 provides schematics showing illustrative embodiments of skin adhesive patches of the invention and systems in which these patches are used. The top panel shows a collapsed/integrated view of a continuous glucose monitoring (CGM) device disposed in vivo, and the bottom panel shows an exploded view of this device illustrating therapeutic agent and adhesive patch elements. Aspects and embodiments of the invention shown in this figure include a glucose sensor coupled to elements including various compositions useful as adhesive agents, therapeutic agents and crosslinking agents. Illustrative adhesive acrylate monomers that are biocompatible and useful in embodiments of the invention include: Hema (Hydroxyethylmethacrylate), MMA (methyl methacrylate), Hydroxybutyl methacrylate, 2-ethylhexyl acrylate, isooctyl acrylate and n-butyl acrylate. Illustrative corticosteroids include: hydrocortisone, fluocinolone acetonide, mometasone furoate, dexamethasone and dexamethasone acetate. Illustrative crosslinkers include EGDA (Ethylene glycol diacetate), poly(ethylene glycol) diacrylate with low molecular weight (e.g. 250), and (3-Aminopropyl)triethoxysilane.

FIG. 12 provides schematics showing illustrative embodiments of skin adhesive patches of the invention and systems in which these patches are used. The top panel shows a collapsed/integrated view of a continuous glucose monitoring (CGM) device disposed in vivo, and the bottom panel shows an exploded view of this device illustrating therapeutic agent and pressure sensitive adhesive patch elements incorporated having a therapeutic agent (corticosteroid) disposed therein. Aspects and embodiments of the invention shown in this figure include a glucose sensor coupled to elements including various compositions useful as adhesive agents and therapeutic agents.

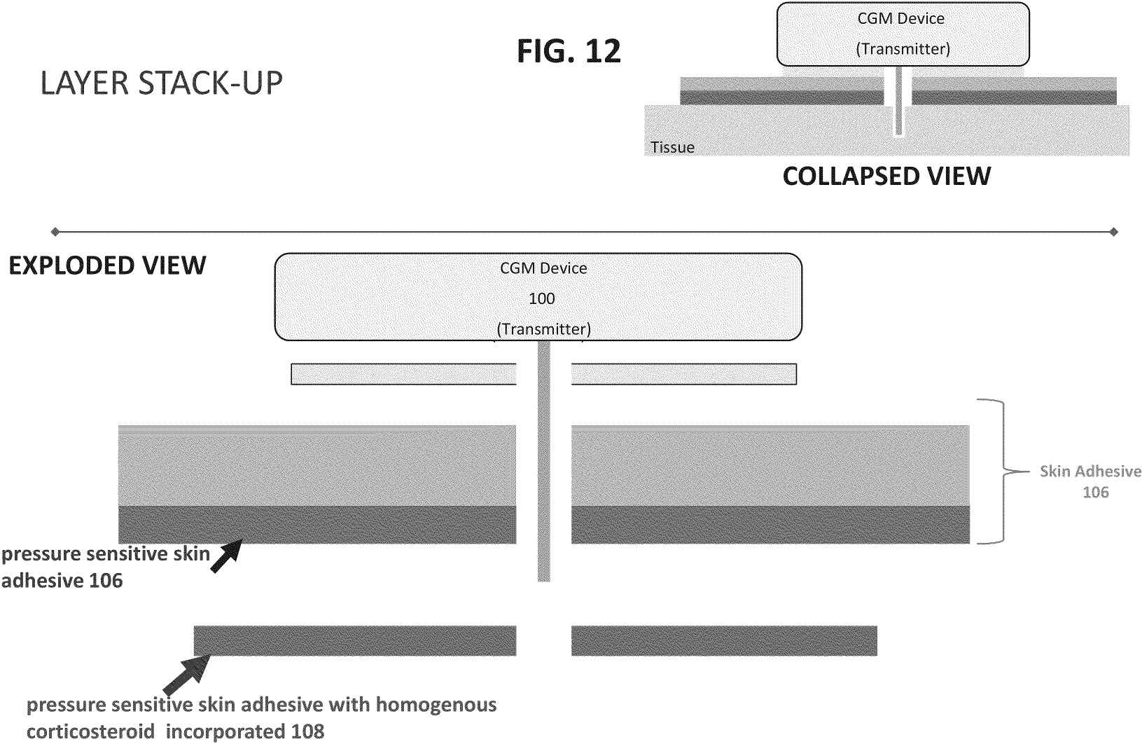

FIG. 13 provides schematics showing illustrative embodiments of skin adhesive patches of the invention comprising acrylic and/or polyurethane materials. The left panel shows the acrylate polymer structures in acrylic based adhesive polymers and the structures of illustrative therapeutic agents, and the right panel shows a backing layer formed from a fiber/polymer nonwovenmesh.

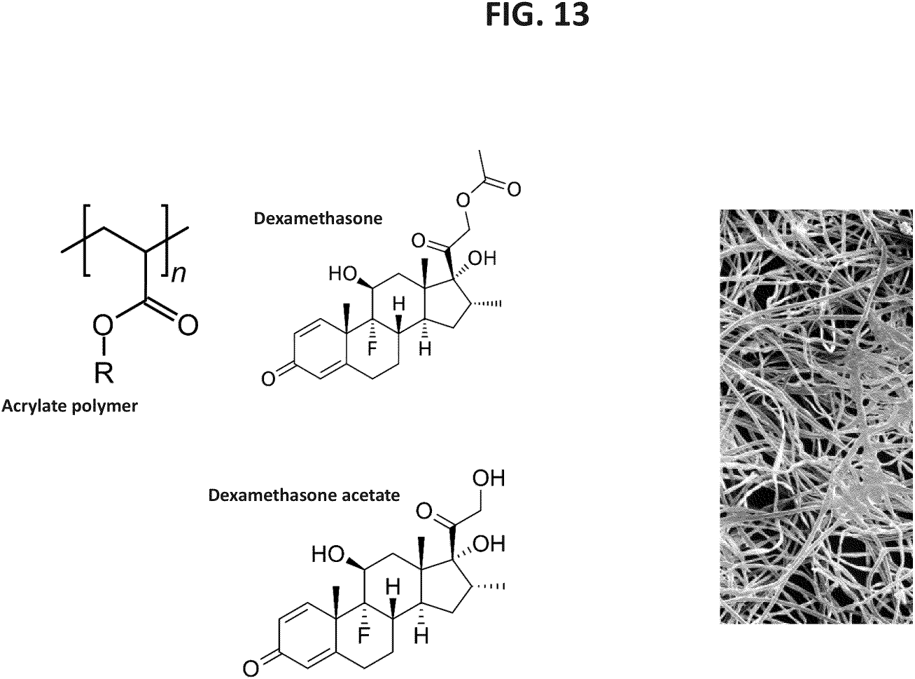

### DETAILED DESCRIPTION OF THE INVENTION

Unless otherwise defined, all terms of art, notations, and other scientific terms or terminology used herein are intended to have the meanings commonly understood by those of skill in the art to which this invention pertains. In some cases, terms with commonly understood meanings may be defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted.

Embodiments of the invention comprise adhesive patches that are adapted to couple medical components (e.g., glucose sensors, infusion sets, infusion pumps and the like) to the skin of patients. Embodiments of the invention can be used with a wide variety of medical components/devices such as glucose sensors and/or insulin infusion sets and insulin pumps that are commonly worn by diabetic patients. In the text below, a continuous glucose monitor comprising a glucose sensor is used as an example of medical components/devices in illustrative embodiments of the invention discussed herein.

The invention disclosed herein has a number of embodiments. Embodiments of the invention include, for example, an adhesive patch comprising a first flexible layer of material adapted to contact skin, an adhesive composition operatively coupled to the first flexible layer of material, and a therapeutic composition such as a corticosteroid operatively coupled to this first flexible layer of material. In such embodiments, the adhesive composition is adapted to adhere the first flexible layer of material to skin; and the therapeutic compound is operatively coupled to the adhesive patch such that when the first flexible layer of material is adhered to skin, the therapeutic compound is exposed to the skin such that inflammation is inhibited at a site on skin where the adhesive patch is adhered.

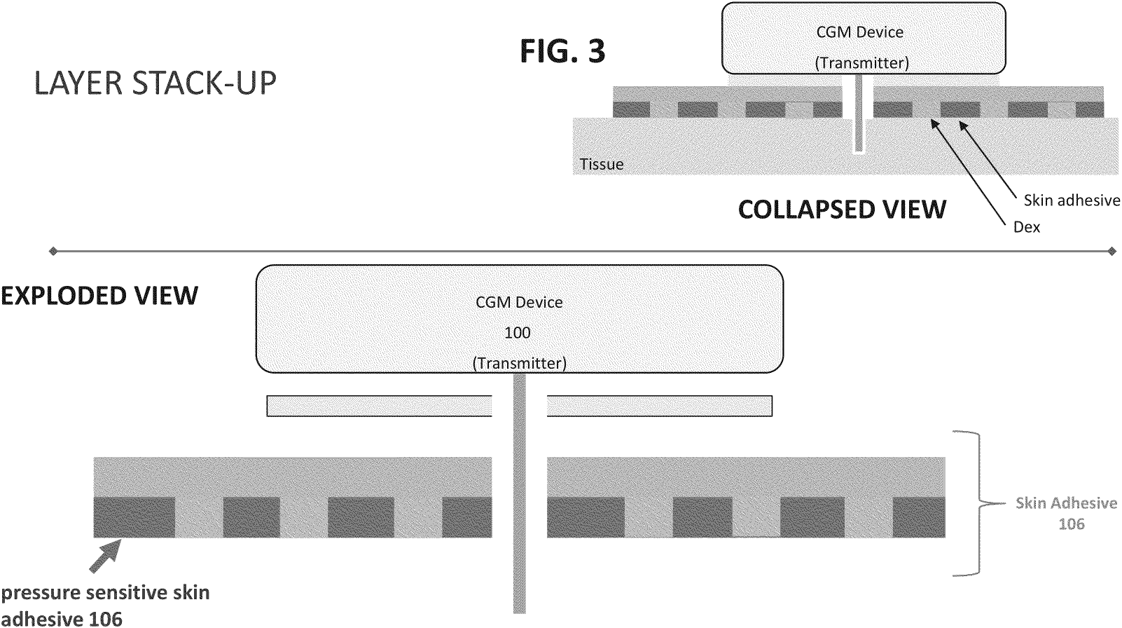

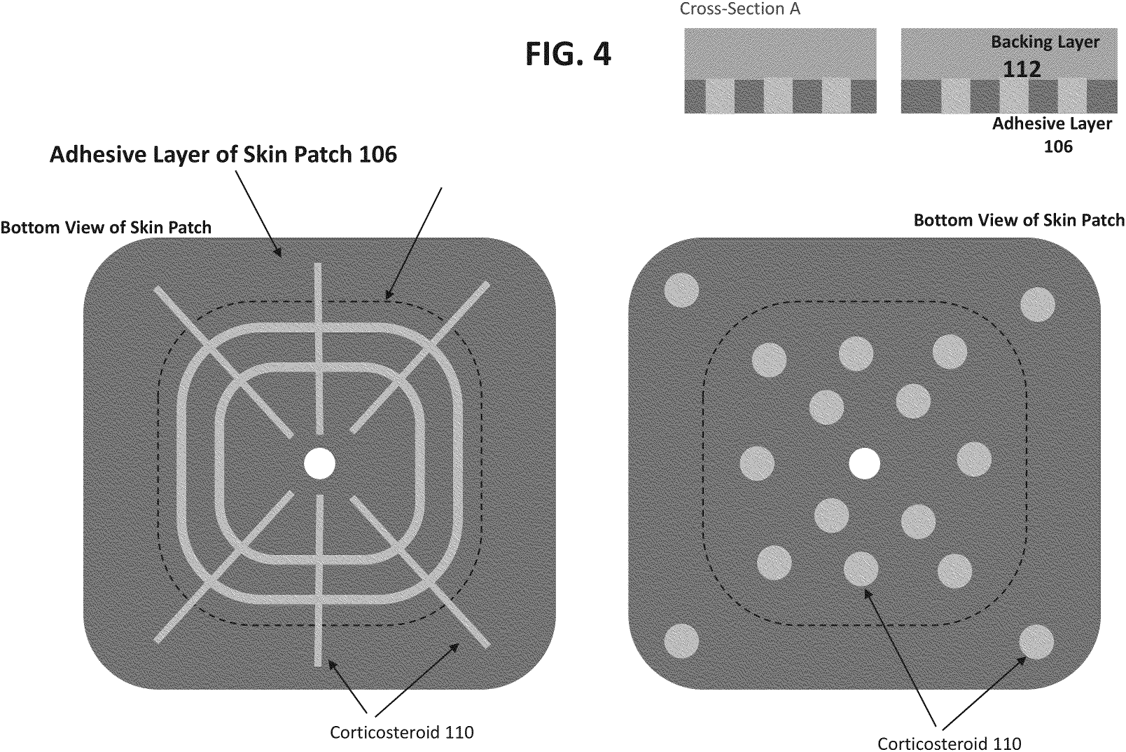

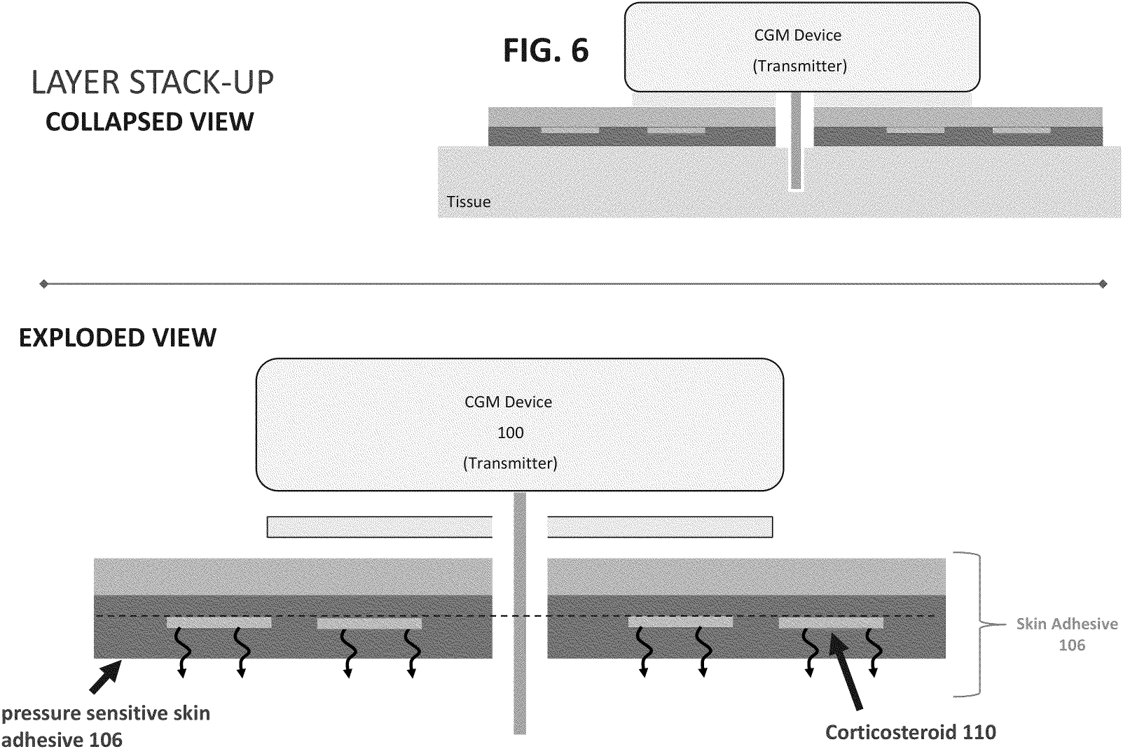

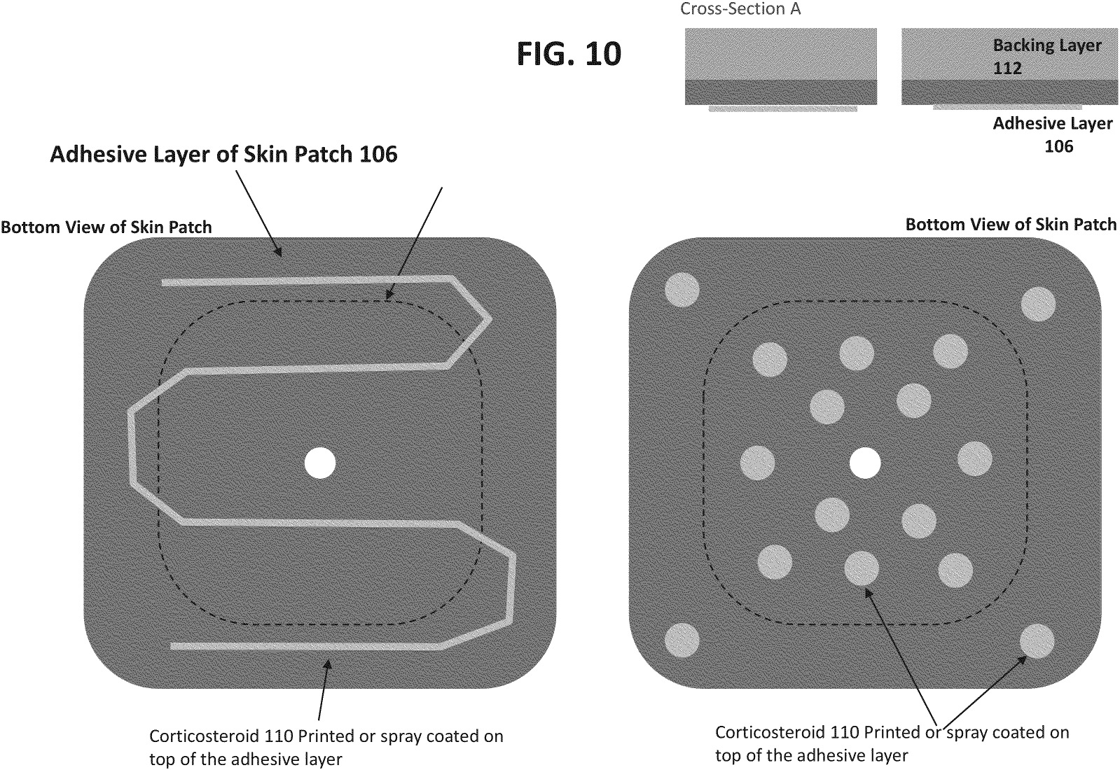

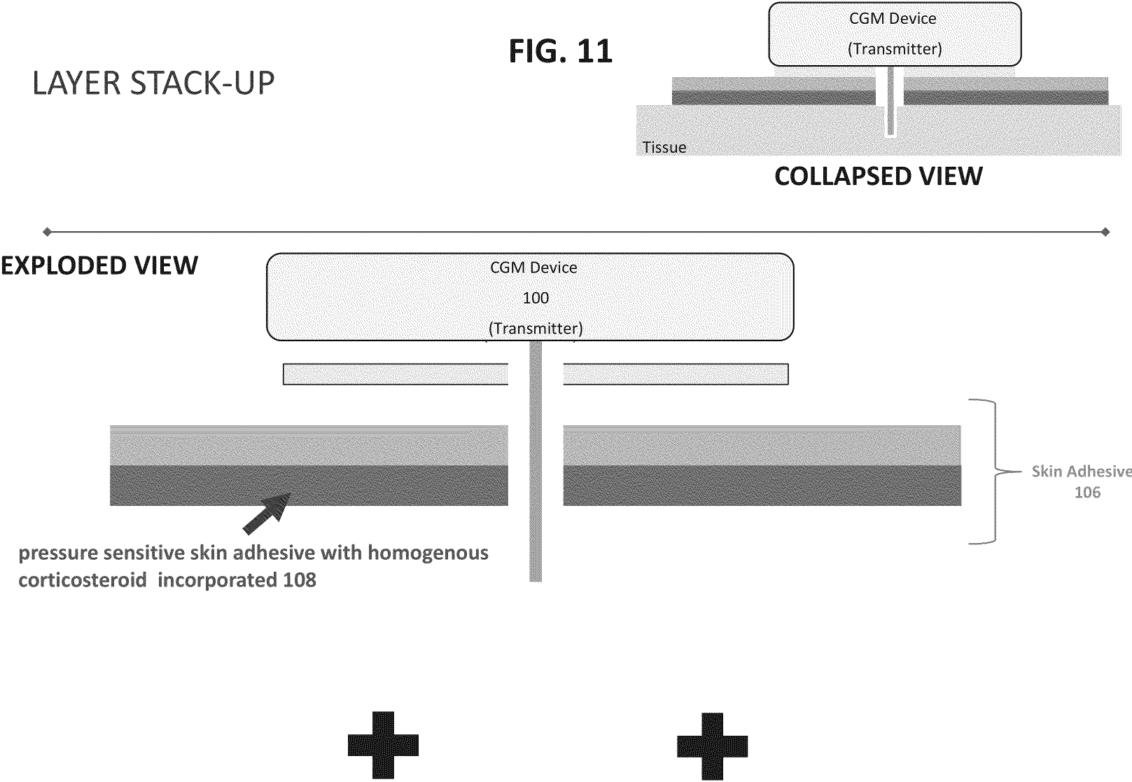

While a wide variety of therapeutic agents can be used with embodiments of the invention, in typical embodiments a corticosteroid is used as the agent (e.g., dexamethasone, dexacetate, prednisone, etc.) and is disposed in the patch so that it is released topically on the skin surface (see, e.g., FIG. 11). In one embodiment the adhesive layer (e.g., one comprising an acrylate composition) is selectively patterned, and the corticosteroid is selectively patterned as shown for example in FIGS. 3-5 and 10). In another embodiment, the corticosteroid is located between the patch backing layer and the acrylate adhesive layer; whereby the corticosteroid diffuses through the acrylate layer to the skin surface (see, e.g., FIG. 6). In another embodiment a thin layer is deposited on the layer on the acrylate surface with a selective pattern (see, e.g., FIG. 4 and FIG. 10).

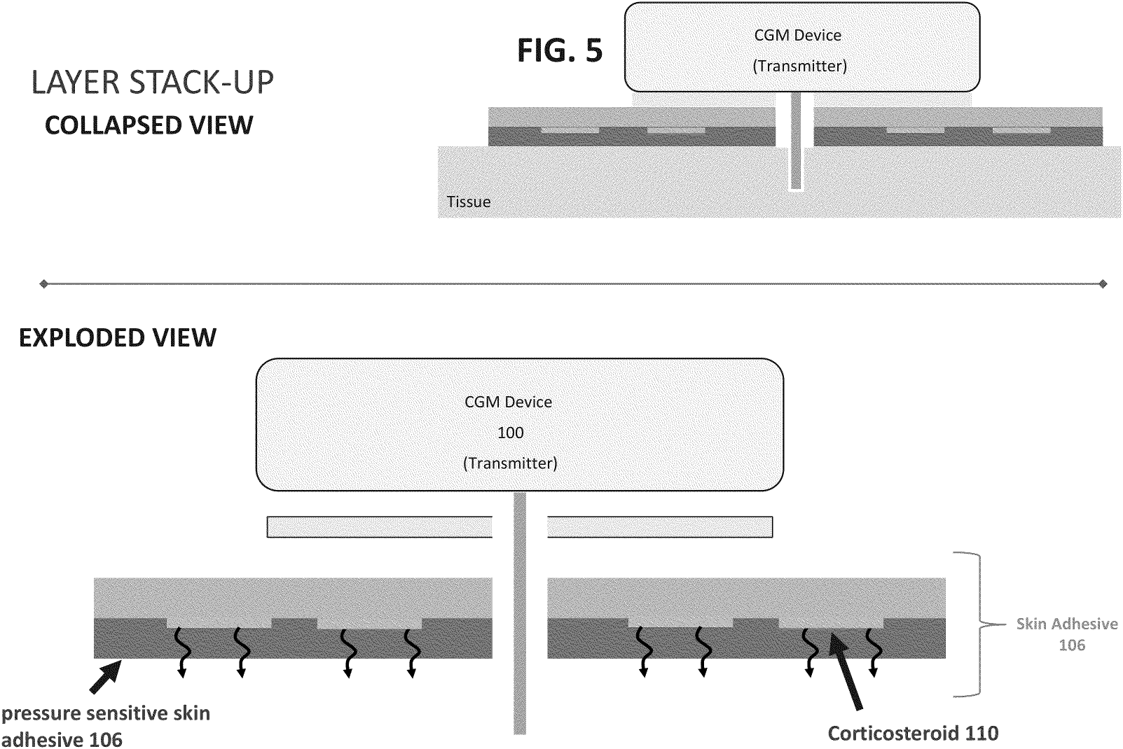

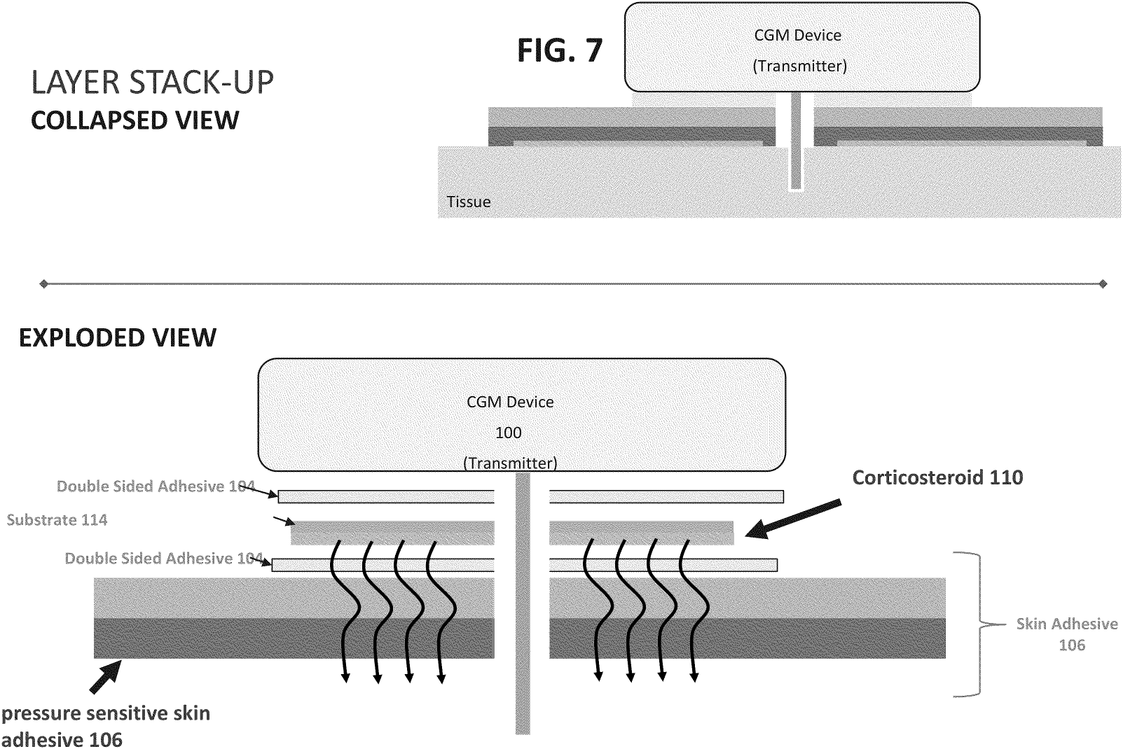

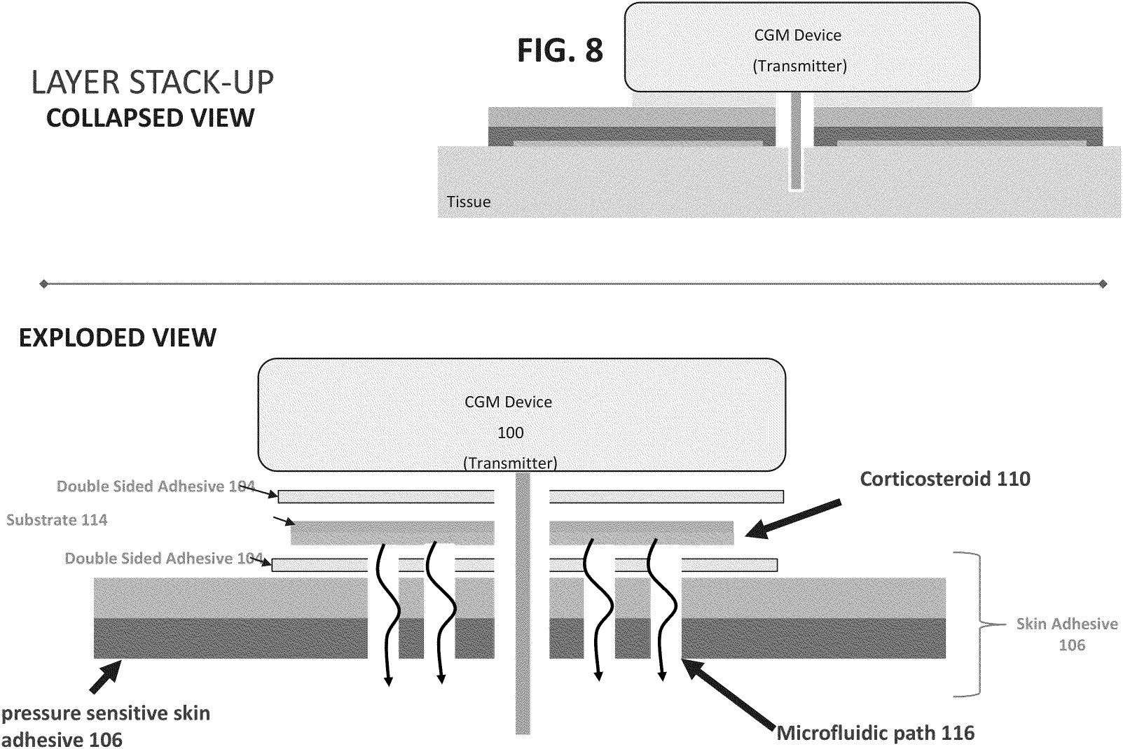

In typical embodiments of the adhesive patch is designed to have elements and/or a three-dimensional architecture that facilitates the therapeutic agent diffusing away from the site at which it is disposed and to the skin of a patient (e.g., via conduits or vias that operably connect the therapeutic compound to skin). Schematics of illustrative embodiments of this are shown in FIGS. 5-8. In some embodiments of the invention, the adhesive composition and/or the therapeutic composition is disposed in the adhesive patch in a selective pattern (e.g, one comprising alternating deposits of adhesive and therapeutic agent as shown in FIGS. 4 and 10). As shown by the schematics of FIGS. 7 and 8, in embodiments of the invention, the adhesive patch can comprise additional elements such as at least one second flexible layer of material (e.g. a second flexible layer also comprising an adhesive composition and which is adapted to adhere the first flexible layer of material to a medical device). In certain embodiments of the invention, the therapeutic compound is disposed on a discrete substrate layer (see, e.g., FIG. 7). Optionally, the therapeutic agent is disposed between the flexible layer of material and the adhesive composition such that the therapeutic agent diffuses through the adhesive composition and to skin; and/or the therapeutic agent is disposed on the adhesive composition such that the therapeutic agent diffuses through the adhesive composition and to skin. In typical embodiments of the invention, the adhesive patch is coupled to a medical device such as a glucose sensor or an infusion set.

Embodiments of the invention include methods of making an adhesive patch as disclosed herein. Typically these methods include operably coupling an adhesive composition operatively coupled to a first flexible layer of material; and operatively coupling a therapeutic composition to the first flexible layer of material. The components of the adhesive patches can be formed using art accepted methods. For example, in methods where the therapeutic agent in dexamethasone, a solution of dexacetate (DXAC) and polyurethane (PU) can be prepared (e.g. 60% PU & 40% DXAC) using with isopropyl alcohol (IPA) + tetrahydrofuran (THF). Dimethylacetamide (DMAC) can also be used as an alternative to IPA + THC. This composition can spray coated on to the desired location/substrate. In such methods, an amount of drug loaded is dependent on the number of spray passes. A mask can be used during the spray coating process to ensure that the agent such as DXAC is deposited only in the desired location (i.e., selective deposition). In typical methods, the adhesive composition is adapted to adhere the first flexible layer of material to skin; and the therapeutic compound is operatively coupled to the adhesive patch such that when the first flexible layer of material is adhered to skin, the therapeutic compound is exposed to the skin such that inflammation is inhibited at a site on skin where the adhesive patch is adhered. In illustrative embodiments of the invention, the therapeutic compound comprises an anti-inflammatory agent such as a corticosteroid and/or the adhesive composition comprises an acrylate. Certain embodiments of the invention include the step of coupling the adhesive patch to a medical device.

The dimensions of the adhesive patch are not critical and may be adapted according to conventional configurations practiced in the art. The patches may be sized as known in the art, with a typical diameter of the patch at the widest point being 1.5 to 2.5 inches, although this dimension likewise is not critical. Apertures, vias and the like through one or more of the layers may be of any shape such as circular, depending on the requirements of receiving a sensor or a cannula, hub or other medical component. The adhesive patches disclosed herein can have additional elements such as tabs adapted to be grasped by the user to manipulate the patch.

Embodiments of the invention further include methods for estimating the concentrations of glucose in vivo, the methods comprising disposing an adhesive patch disclosed herein coupled to a glucose sensor on a diabetic patient; and estimating concentrations of glucose using the glucose sensor. Typically in these methods, the adhesive patch is worn by the patient for at least 10 days, at least 15 days, or at least 20 days. Optionally in these methods, the patch is coupled to an insulin infusion set and/or an infusion pump.

Embodiments of the invention can use pressure sensitive adhesive compositions. Pressure sensitive adhesives (“PSAs”) based on polymers having acrylic or acrylate moieties are well known in the art and have been used for many years in the medical device arts. Pressure-sensitive acrylic adhesives for application to skin are typically made from compounds such as: 2-ethylhexyl acrylate, isooctyl acrylate or n-butyl acrylate. Polymers in such adhesives can be copolymerized with polar functional monomers such as: acrylic acid, methacrylic acid, vinyl acetate, methyl acrylate, N-vinylcaprolactam, or hydroxyethyl methacrylate (see, e.g., Kenney, J. F., et al. “Medical-grade acrylic adhesives for skin contact.” Journal of applied polymer science 45.2 (1992): 355-361). Other PSAs may be based on silicones. The selection of an appropriate PSA having the desired peel strength may be left to the skill of the person of ordinary skill in this art. Peel strength is a measure of how firmly the PSA adheres to the user’s body and may be measured in various ways, measuring the force required to separate two reference surfaces adhered with the PSA. In some embodiments, the PSA is modified by adding one or more active agents, such as a corticosteroid directly to the PSA composition.

As shown in the figures, embodiments of the invention can include a variety of elements such as a continuous glucose monitoring (CGM) device 100, a subcutaneous glucose sensor 102, a double sided pressure sensitive adhesive 104, a skin adhesive layer 106, a skin adhesive layer comprising a therapeutic agent 108, a therapeutic agent 110, a backing layer 112, a substrate 114 and a microfluidic path 11. In embodiments of the invention, a pressure sensitive adhesive layer covers substantially the entire extent of the adhesive surface. Alternatively, regions of the adhesive surface are covered with a pressure sensitive adhesive and adjacent regions are covered with active agents such as a therapeutic agent (see, e.g., FIGS. 3-5 and 10). This configuration is preferable if the active agent is expected to degrade the performance of the pressure sensitive adhesive after prolonged contact. Applying pressure sensitive adhesive in a region distinct from an active agent region also permits the infusion set to be tailored according to the user’s skin sensitivity. For example, the area near an injection site may be especially sensitive, and it may be preferable to limit an active agent, such as a corticosteroid, to an area of the adhesive surface out of contact with the injection site. With therapeutic agents it may be desirable to combine the active agent and the pressure sensitive adhesive in one composition. As would be understood by the person of ordinary skill in the art, some minor area of the adhesive surface could be left uncovered by adhesive or active agent(s), provided the functioning of the patch is not deleteriously impacted. While a wide variety of adhesives can be used in such patches, in illustrative embodiments of the invention, the adhesive composition comprises an acrylate.

While a wide variety of therapeutic agents can be used in such patches, typically in these embodiments, the therapeutic compound comprises an anti-inflammatory agent such as a corticosteroid. Examples of anti-inflammatory drugs include both steroidal and non-steroidal (NSAID) anti-inflammatories such as, without limitation, clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cortodoxone, deflazacort, desonide, desoximetasone,momentasone, cortisone, cortisone acetate, hydrocortisone, prednisone, prednisone acetate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, tacrolimus and pimecrolimus.

Additionally, examples of steroidal anti-inflammatory drugs include, without limitation, 21-acetoxypregnenolone, alclometasone, algestone, amcinonide, budesonide, chloroprednisone, clobetasol, clobetasone, clocortolone, cloprednol, corticosterone, cortisone, cortivazol, deflazacort, desonide, desoximetasone, diflorasone, diflucortolone, difluprednate, enoxolone, fluazacort, flucloronide, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, any of their derivatives, and combinations thereof.

Furthermore, examples of nonsteroidal anti-inflammatory drugs include, without limitation, COX-1 and COX nonspecific inhibitors (e.g., salicylic acid derivatives, aspirin, sodium salicylate, choline magnesium trisalicylate, salsalate, diflunisal, sulfasalazine and olsalazine; para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin), and selective COX-2 inhibitors (e.g., diaryl-substituted furanones such as rofecoxib; diaryl-substituted pyrazoles such as celecoxib; indole acetic acids such as etodolac and sulfonanilides such as nimesulide), and combinations thereof.

Additionally, other naturally occurring or synthetic drugs, agents, molecules (e.g., hyaluronidase), and proteins may be included with the response-inhibiting agent to mitigate foreign-body responses and/or help facilitate the body in absorbing the medication.

It is to be understood that this invention is not limited to the particular embodiments described, as such may, of course, vary. It is also to be understood that the terminology used herein is for the purpose of describing particular embodiments only, and is not intended to be limiting, since the scope of the present invention will be limited only by the appended claims. In the description of the preferred embodiment, reference is made to the accompanying drawings which form a part hereof, and in which is shown by way of illustration a specific embodiment in which the invention may be practiced. It is to be understood that other embodiments may be utilized, and structural changes may be made without departing from the scope of the present invention.

1. An adhesive patch comprising:
a first flexible layer of material adapted to contact skin;
an adhesive composition operatively coupled to the first flexible layer of material; and
a therapeutic composition operatively coupled to the first flexible layer of material; wherein:
the adhesive composition is adapted to adhere the first flexible layer of material to skin; and
the therapeutic compound is operatively coupled to the adhesive patch such that when the first flexible layer of material is adhered to skin, the therapeutic compound is exposed to the skin such that inflammation is inhibited at a site on skin where the adhesive patch is adhered.

2. The adhesive patch of claim 1, wherein:
the therapeutic compound comprises an anti-inflammatory agent such as a corticosteroid;
the adhesive composition comprises an acrylate; and/or
the adhesive patch comprises vias that operably connect the therapeutic compound to skin.

3. The adhesive patch of claim 1, wherein the adhesive composition and/or the therapeutic composition is disposed in the adhesive patch in a selective pattern comprising .

4. The adhesive patch of claim 1, wherein the adhesive patch comprises at least one second flexible layer of material:.

5. The adhesive patch of claim 4, wherein the second flexible layer of material comprises an adhesive composition and is adapted to adhere the first flexible layer of material to a medical device.

6. The adhesive patch of claim 1, wherein the adhesive patch is coupled to a medical device.

7. The adhesive patch of claim 6, wherein the medical device is a glucose sensor.

8. The adhesive patch of claim 1, wherein the therapeutic compound is disposed on a discrete substrate layer.

9. The adhesive patch of claim 1, wherein:
the therapeutic agent is disposed between the flexible layer of material and the adhesive composition such that the therapeutic agent diffuses through the adhesive composition and to skin; and/or
the therapeutic agent is disposed on the adhesive composition such that the therapeutic agent diffuses through the adhesive composition and to skin.

10. A method of making an adhesive patch comprising:
operably coupling an adhesive composition operatively coupled to a first flexible layer of material; and
operatively coupling a therapeutic composition to the first flexible layer of material; wherein:
the adhesive composition is adapted to adhere the first flexible layer of material to skin; and
the therapeutic compound is operatively coupled to the adhesive patch such that when the first flexible layer of material is adhered to skin, the therapeutic compound is exposed to the skin such that inflammation is inhibited at a site on skin where the adhesive patch is adhered.

11. The method of claim 11, wherein:
the therapeutic compound comprises an anti-inflammatory agent such as a corticosteroid;
the adhesive composition comprises an acrylate; and/or
the adhesive patch is formed to comprise vias that operably connect the therapeutic compound to skin.

12. The method of claim 11, wherein the adhesive patch is formed to comprise at least one second flexible layer of material:.

13. The method of claim 12, wherein the second flexible layer of material is formed to comprise an adhesive composition and is adapted to adhere the first flexible layer of material to a medical device.

14. The method of claim 11, further comprising coupling the adhesive patch to a medical device.

15. A method of sensing glucose comprising:
disposing the adhesive patch of claim 7 on a diabetic patient; and
sensing glucose using the glucose sensor.

16. The method of claim 15, wherein the adhesive patch is worn by the patient for at least 10 days, at least 15 days, or at least 20 days.

17. The method of claim 15, wherein the patch is coupled to an insulin infusion set and/or an infusion pump.

18. The method of claim 15, wherein the therapeutic compound comprises a corticosteroid selectively patterned on the patch.
